(19) United States
(12) Patent Application Publication
(10) Pub. No.: US 2017/0066835 A1
(43) Pub. Date: Mar. 9, 2017

(54) PHARMACEUTICAL COMPOSITION COMPRISING AN ANTI-CD6 MONOCLONAL ANTIBODY USED IN THE DIAGNOSIS AND TREATMENT OF RHEUMATOID ARTHRITIS

(71) Applicant: CENTRO DE INMUNOLOGIA MOLECULAR, CIUDAD DE LA HABANA (CU)

(72) Inventors: JOSE ENRIQUE MONTERO CASIMIRO, C. HABANA (CU); ANGEL RAIMUNDO CASACO PARADA, C. HABANA (CU); ZAIMA MAZZORA HERRERA, C. HABANA (CU); RUBY ALONSO RAMIREZ, HABANA (CU); ROLANDO PEREZ RODRIGUEZ, C. HABANA (CU)

(21) Appl. No.: 15/352,982

(22) Filed: Nov. 16, 2016

Related U.S. Application Data
(62) Division of application No. 14/252,002, filed on Apr. 14, 2014.
Application No. 14/252,002 is a division of application No. 12/525,442, filed on Dec. 9, 2009.

(30) Foreign Application Priority Data
Dec. 26, 2006 (CU) 2006-0250

(51) Int. Cl. C07K 16/28; A61K 45/06; A61K 39/395

(52) U.S. Cl. CPC C07K 16/2896; A61K 39/3955; A61K 45/06; C07K 2317/14; C07K 2317/24; A61K 2039/505

(57) ABSTRACT

The present invention is related to the branch of immunology and particularly with the generation of pharmaceutical compositions containing a humanized monoclonal antibody recognizing the leukocyte differentiation antigen CD6. Accordingly with that statement, the purpose of this invention is to provide pharmaceutical compositions which contain a humanized anti-monoclonal antibody for the diagnosis and treatment of Autoimmune Diseases, particularly the Rheumatoid Arthritis.

### CROSS-REFERENCE TO RELATED APPLICATIONS

This application is a continuation application of copending U.S. patent application Ser. No. 14/252,002, filed on Apr. 14, 2014, which in turn is a divisional application of copending U.S. patent application Ser. No. 12/525,442 filed on Dec. 9, 2009 and claiming priority thereto which in turn is an application filed under 35 U.S.C. §371 claiming priority of International Patent Application No. PCT/CU2007/000021 filed on Dec. 24, 2007, which in turn claims priority of Cuban Application No. 2006-0250 filed on Dec. 26, 2006, the contents of which are incorporated by reference herein for all purposes.

### FIELD OF THE INVENTION

The present invention is related to the human medicine and especially with the generation of pharmaceutical compositions comprising a humanized monoclonal antibody recognizing the leukocyte differentiation antigen CD6, and particularly with therapeutic formulations which contain a humanized monoclonal antibody that recognizes the leukocyte differentiation antigen CD6 able to induce a long-lasting therapeutic effect in Rheumatoid Arthritis patients.

### DESCRIPTION OF THE PRIOR ART

Autoimmune Diseases and particularly, Rheumatoid Arthritis (RA) have available different strategies with therapeutic purposes, associated to their known immunopathologic mechanisms (Smolen, J. S. et al. (2003) Nature Reviews Drug Discovery 2:473; Feldmann, M. et al. (2005) Nature 435:612). However, there is not a treatment for Rheumatoid Arthritis that administered for a short period of time may induce a long-lasting clinical response (Garber, K. (2005) Nature Biotechnology 23 (11): 1323).

Several biotherapies are currently available for the treatment of patients with Autoimmune Diseases and particularly for therapy of Rheumatoid Arthritis patients, although their clinical effect as monotherapies is very limited (Olsen, N. J. et al. (2004) N Engl J Med 350:2167). Moreover, combinatorial therapies of biological agents with Methotrexate may induce clinical remission in 30-40% of Rheumatoid Arthritis patients, but the disease remains significantly active in most of the patients despite the treatment and complete remissions rarely occur (Pincus, T. et al. (1999) Ann Intern Med 131:768; Olsen, N. J. et al. (2004) N Engl JMed 350:2167). Additionally, multiple and severe adverse events have been described for such therapeutic approximations (O'Dell, J. R. (2004) N Engl J Med 350:2591).

Monoclonal antibodies (mAbs) are biological agents used in the treatment of Autoimmune Diseases (Feldmann, M. et al. (2005) Nature 435:612). Particularly, monoclonal antibodies depleting with the capacity to deplete autoreactive cells are of medical interest due to the consideration that a prolong depletion of lymphocytes is required to induce a therapeutic effect in Autoimmune Disease patients, such as Rheumatoid Arthritis (Edwards, J. C. et al. (2004) N Engl J Med 350:2572; Stohl, W. et al. (2006) Clinical Immunology 121:1; Browning, J. L. (2006) Nature Reviews Drug Discovery 5:564). This immunosuppression induced by lymphocyte depletion is a common mechanism of therapeutic effect for several therapeutic options in Autoimmune Diseases (Kahan, B. D. (2003) Nature Reviews Immunology 3:831). Nevertheless, therapeutic effects are limited and associated to significant adverse events (Edwards, J. C. et al. (2004) N Engl J Med 350:2572; Hale, D. A. (2004) Current Opinion Immunology 16:565; Goldblatt, F. et al. (2005) Clinical and Experimental Immunology 140:195).

Consequently, a better understanding of molecules and mechanisms involved in the physiopathology of Autoimmune Diseases, and particularly in Rheumatoid Arthritis, should lead to the discovery of new therapeutic targets where the intended therapy switch from the elimination of autoreactive cells to the induction or the restoration of the mechanism of tolerance and immunoregulation in the patient (Taylor, P. C. et al. (2004) Current Opinion Pharmacology 4:368; Feldmann, M. et al. (2005) Nature 435:612).

The leukocyte differentiation antigen CD6 is a molecule not well studied and characterized yet. The CD6 is a surface glycoprotein expressed primarily in T lymphocytes and in a minor subpopulation of B lymphocytes in peripheral blood of normal individuals. However the origin and the functional characterization in these cells are very limited. Basically, it is considered that CD6 in these cells is a receptor with co-stimulatory function, but the mechanism in unknown (Aruffo, A. et al. (1997) Immunol Today 18(10):498; Patel, D. D. (2000) J Biol Regul Homeost Agents 2000 14(3):234). Its expression in mature thymocytes has been associated to the lymphocyte maturation process in this lymphoid organ (Singer, N. G. et al. (2002) Int Immunol. 14(6):585).

The CD6 model has been of therapeutic interest and it has been claimed that the mechanism of action for anti-CD6 monoclonal antibodies is based in their capability to inhibit and modulate the CD6 binding to its ligand named ALCAM (Activated Leukocyte Adhesion Molecule) (U.S. Pat. No. 6,372,215). Consequently, those monoclonal antibodies are considered useful for the treatment of Autoimmune Diseases, but therapeutic evidences in patients based on such claim are not substantially documented.

CD6 molecule is recognized by the mouse monoclonal antibody ior-t1. Therapeutic formulations of this murine monoclonal antibody have therapeutic effect in Psoriasis (Montero, E. et al. (1999) Autoimmunity 29(2):155). Subsequently, based on methods of genetic engineering (Pat. No. 0,699,755 E.P. Bul.) it was obtained a humanized version of this mouse anti-human CD6 monoclonal antibody designated T1h (EP 0 807 125 A2).

### SUMMARY OF INVENTION

The novelty of this invention relies on the generation of therapeutic formulations containing anti-CD6 monoclonal antibodies for their use in Autoimmune Disease patients and particularly, in Rheumatoid Arthritis patients. Surprisingly, the administration of the humanized T1h monoclonal antibody during 6 weeks as a monotherapy, in Autoimmune Disease patients and particularly in Rheumatoid Arthritis patients with a prolonged evolution and in active phase of the disease induce a long-lasting therapeutic effect lasting for months after finishing the administration of the treatment, without significant adverse events. This effect is not associated to the depletion of the CD6+ cells because the recovery to the normal value of that cellular subpopulation does not impede a persistent clinical improvement of the disease. The humanized T1h monoclonal antibody does not inhibit the binding of CD6 to the ALCAM, effect that was previously claimed for therapies in this therapeutic model, indicating its potential association to an alternative mechanism.

Additionally, T1h treatment may sensitize autoimmune cells to the effect of anti-inflammatory agents, steroids or chemotherapies as Methotrexate, which may lead to the combinatorial use of humanized monoclonal antibody T1h with other drugs or other biotherapies.

### BRIEF DESCRIPTION OF THE FIGURES

FIG. 1.—Therapeutic effect of the humanized monoclonal antibody T1h in Rheumatoid Arthritis patients evaluated by the reduction of the percent of swollen and tender joints.

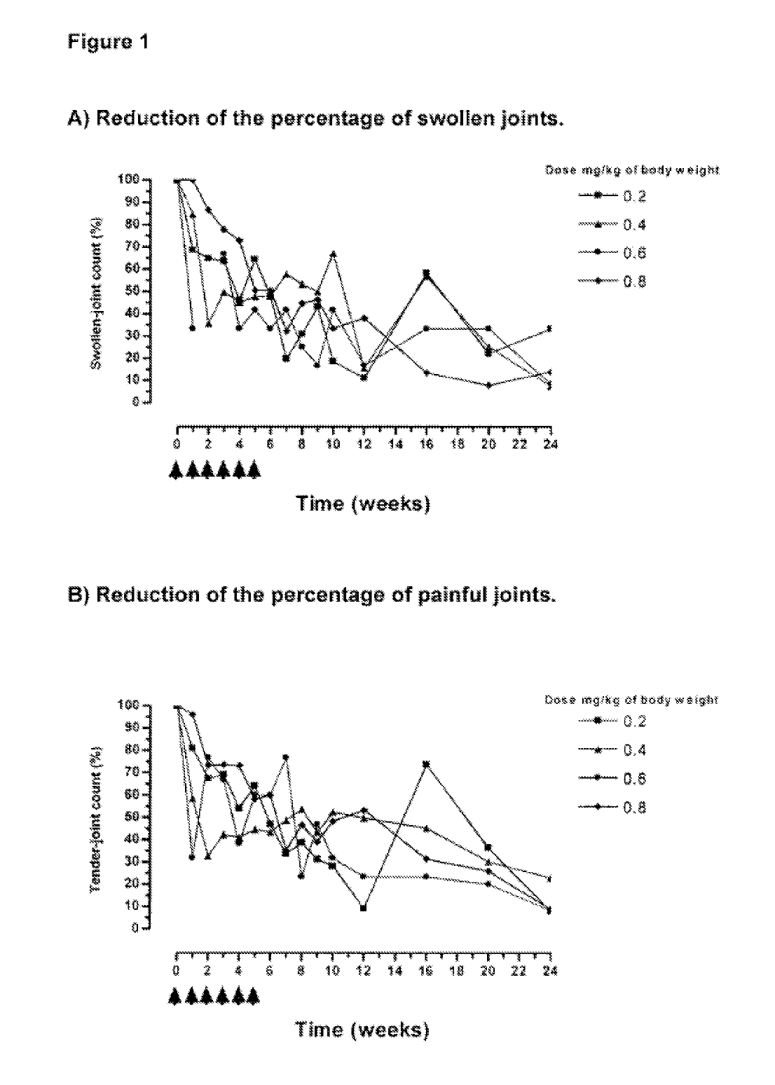

FIG. 2.—Quantification of the number of lymphocytes CD3+ and CD6+ in peripheral blood mononuclear cells from Rheumatoid Arthritis patients

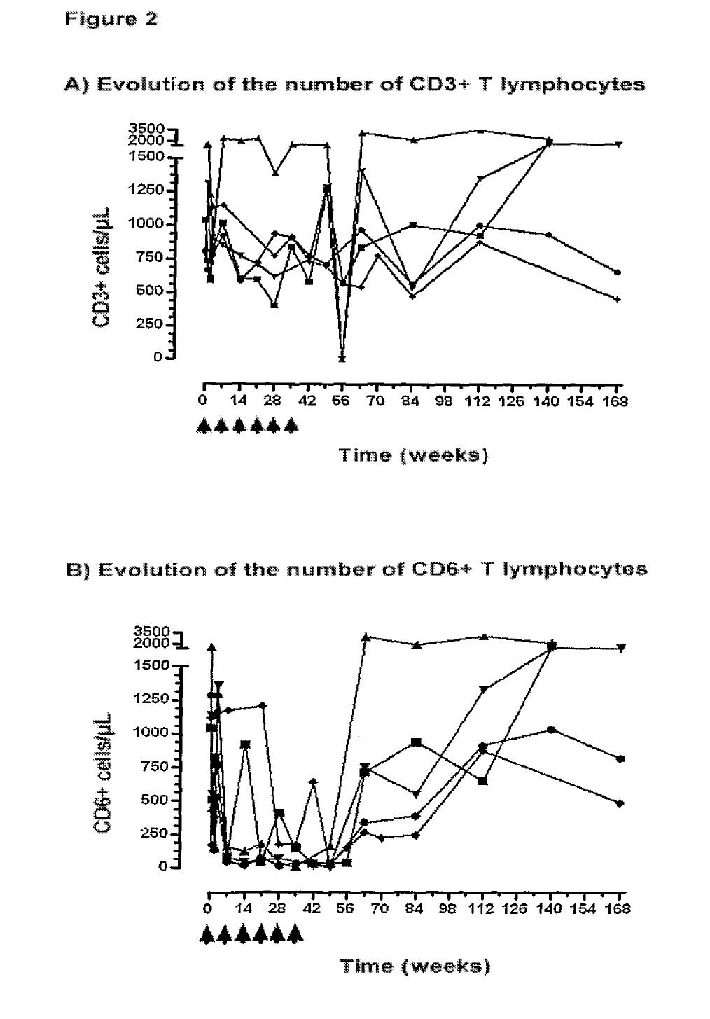

FIG. 3.—Demonstration of the non-inhibition of the CD6 binding to the ALCAM ligand by the humanized monoclonal antibody T1h

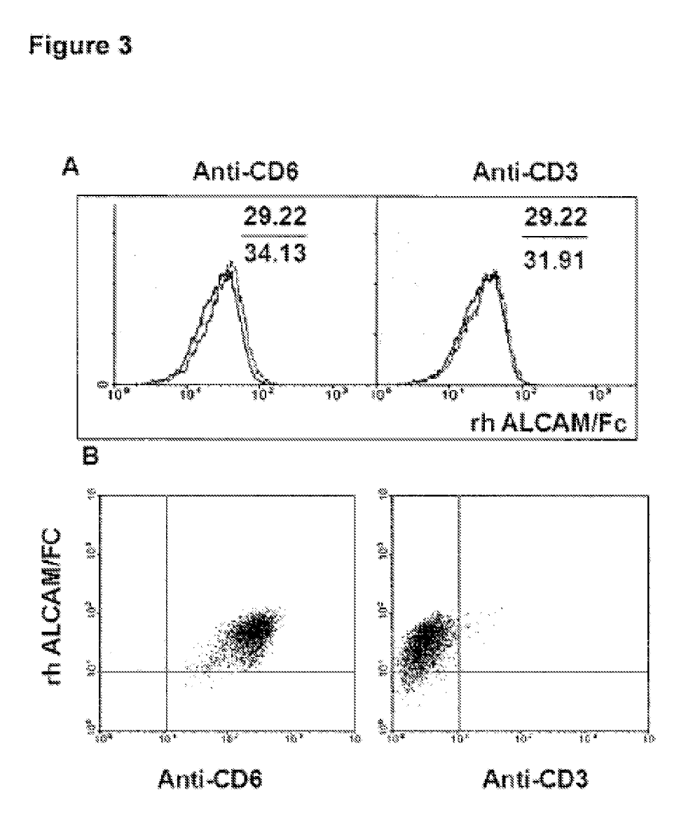

### DETAILED DESCRIPTION OF THE INVENTION

The present invention relates with therapeutic formulations of monoclonal antibodies that recognize the human antigen CD6, effective in the treatment of patients with Autoimmune Diseases. More particularly, the present invention comprises the use of pharmaceutical compositions containing the humanized monoclonal antibody T1h, which recognizes the human leukocyte differentiation antigen CD6 and their use for the diagnosis and treatment of the Rheumatoid Arthritis.

It is a subject of the present invention a therapeutic formulation containing the humanized monoclonal antibody T1h, which induces a long-lasting therapeutic effect in Rheumatoid Arthritis patients by the recognition of the CD6 molecule. The term “humanized Monoclonal Antibody” refers to a monoclonal antibody obtained by genetic engineering methods as described in the (Pat. No. 0,699,755 (E. P. Bul).

1.—Generation of Pharmaceutical Compositions Contains the Humanized Anti-Human CD6 Monoclonal Antibody T1h.

The pharmaceutical composition of the present invention contains a humanized monoclonal antibody T1h that recognizes the human leukocyte differentiation antigen CD6. The humanized anti-human CD6 monoclonal antibody T1h is obtained from the secreting hybridoma IO-T1A with deposit No. ECACC 96112640, as described in (EP 0 807 125 A2). Additionally, this composition contains as an appropriate excipient a physiological buffered solution, similar to others used for the formulation of monoclonal antibodies for intravenous use, as described in EP 0807125. The composition of the present invention is administered in the way of injections in a range of doses from 0.05 to 1 mg/Kg of body weight

2.—Characterization of the Therapeutic Effect of the Pharmaceutical Compositions Containing the Humanized Anti-human CD6 Monoclonal Antibody T1h.

Rheumatoid Arthritis patients diagnosed according to the standard criteria and in an active phase of the disease, resistant to conventional therapies, such as anti-inflammatory drugs, steroids or chemotherapeutic agents (e.g.: Methotrexate) are susceptible to the administration of the humanized monoclonal antibody T1h as monotherapy or in combination with anti-inflammatory drugs, steroids, chemotherapeutic agents (e.g.: Methotrexate) or monoclonal antibodies specific to surface molecules of T and B lymphocytes (e.g.: CD20) or cytokines (e.g.: Tumor Necrosis Factor alpha). The humanized monoclonal antibody T1h may be administered as a parenteral solution in a range of doses according to the body weight of the patient, with a variable frequency of administration which may comprise a daily, weekly or for a longer period administration. The therapeutic effect is evaluated by the reduction of the clinical activity of the disease, according to the standard criteria before, during and after finishing the treatment.

3.—Characterization of the Effect on Peripheral Blood Mononuclear Cells of Pharmaceutical Compositions Containing the Humanized Monoclonal Antibody T1h. Peripheral blood mononuclear cells from Rheumatoid Arthritis patients are incubated with an anti-CD3 or an anti-CD6 monoclonal antibody conjugated to biotin or fluorescent substances (e.g.: FITC, PE or PE-Cy5). The binding of biotinylated antibodies is detected with a Streptavidin, PE-Cy5.5 conjugate. At least 10 000 living cells are acquired in a Flow Cytometer FACScan. Dead cells are excluded with the Propidium Iodine staining.

4.—Characterization of the Ability of the Pharmaceutical Compositions Containing the Humanized Monoclonal Antibody T1h to Inhibit the CD6 Binding to its Ligand ALCAM.

The human epithelial cell line HEK-293 transfected with the human molecule CD6 is incubated with saturated concentration of the anti-human CD6 monoclonal antibody T1h or an isotype control, an anti-human CD3 antibody during 30 minutes at 4° C. Cells are washed and incubated with 0.5 μg/mL of the fusion protein rhALCAM-Fc (ALCAM is a CD6 ligand) for 30 min at 4° C. Then, samples are stained with an anti-human IgG FITC-antibody conjugated for 30 min at 4° C. Ten thousand living cells are acquired in a Flow Cytometer FACScan. Dead cells are excluded with the Propidium Iodine staining.

### EXAMPLES

The humanized anti-human CD6 Monoclonal Antibody T1h was obtained from the hybridoma IOR-T1A with deposit No. ECACC 96112640, as described in EP 0 807 125 A2.

### Example 1

The Humanized Monoclonal Antibody T1h Induce a Long-Lasting therapeutic effect in Rheumatoid Arthritis patients

The therapeutic effect of the humanized Monoclonal Antibody T1h was evaluated or assessed in 13 Rheumatoid Arthritis patients. Patients received a weekly dose of the humanized monoclonal antibody T1h during 6 weeks in a range of doses of 0.2, 0.4, 0.6 and 0.8 mg/Kg of body weight. The therapeutic effect was evaluated by the reduction of the clinical activity of the disease, considering the number of affected joints according to the standard criteria before, during and after finishing the treatment. Each curve represents the mean values of the percentage of improvement of the clinical sign or symptom per group of patient according to the administered dose.

### Example 2

The Treatment with the Humanized Monoclonal Antibody T1h Transiently Reduces the Number of Peripheral Blood Mononuclear Cells from Rheumatoid Arthritis Patients

Peripheral blood mononuclear cells from Rheumatoid Arthritis patients treated with the humanized monoclonal antibody T1h were analyzed. The expression of the CD3 molecule, as a distinctive T lymphocyte marker, as well as the CD6 molecule was determined. The humanized monoclonal antibody T1h treatment induces a transient reduction of CD3+ and CD6+ lymphocytes. However, a recovery to the normal values does not influence the persistent clinical improvement of the disease. The study was performed by flow cytometry using a FACScan to analyze the samples. Each curve represents the values of individual patients in different time points.

### Example 3

The Humanized Monoclonal Antibody T1h does not Inhibit the CD6 Binding to its Ligand ALCAM

The capacity of the humanized monoclonal antibody T1h to inhibit the binding of ALCAM to the human epithelial cell line HEK-293, transfected with the human CD6 molecule was evaluated. (A) Red histogram: recognition of the human recombinant protein ALCAM bound to a human Fc fragment (rhALCAM-Fc) pre-incubated with the anti-CD6 (T1h) or anti-CD3 (control) antibodies; Black histogram: binding of the rhALCAM-Fc to non-treated cells; and Grey Histogram: labeled cells with the FITC-conjugated anti-human IgG antibody. The mean fluorescence intensity values are depicted in the figure. (B) Dot plots show the double staining for rhALCAM-Fc and anti-CD6 or anti-CD3 antibodies.

That which is claimed is:

1. A composition comprising an antiCD6 monoclonal antibody that recognizes human leukocyte differentiation antigen CD6 and does not inhibit the binding of CD6 to ALCAM, wherein the antiCD6 monoclonal antibody is T1h and produced by secreting hybridoma IOR-T1A deposited with the ECACC as deposit No. ECACC 96112640, and wherein the antiCD6 monoclonal antibody is in a therapeutic amount to cause at least 60% depletion of CD6+ lymphocytes within about 7 weeks in a subj ect.

2. The composition of claim 1 further comprising a pharmaceutical carrier.

3. The composition of claim 1, further comprising an anti-inflammatory agent.
